(19) United States
(12) Patent Application Publication
(10) Pub. No.: US 2019/0274874 A1
(43) Pub. Date: Sep. 12, 2019

(54) TRANSFORMATION CHAMBER

(71) Applicant: ANTHONY MICHAEL GUARNIERI, LOS ANGELES, CA (US)

(72) Inventor: ANTHONY MICHAEL GUARNIERI, LOS ANGELES, CA (US)

(21) Appl. No.: 15/915,426

(22) Filed: Mar. 8, 2018

(51) Int. Cl. A61F 7/00

(52) U.S. Cl. CPC A61F 7/0053; F25D 17/005; A61F 2007/0064

(57) ABSTRACT

The Transformation Chamber is unique in that it combines three additional elements to a regular cryotherapy session and is the last portion of the session. The Transformation Chamber has infrared and near infrared lighting that works in-conjunction with a guided meditation, and vibration. With all elements combined in one device allows client's to achieve more in less time in one place.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING

Not applicable

### BACKGROUND OF INVENTION

The idea of The Transformation Chamber came to me while I was thinking about how I've personally transformed my life for the better. I've read many self help books and have always been into health and wellness. So The Transformation Chamber helps to heal people holistically using a combination of element's.

### BRIEF SUMMARY OF INVENTION

The Transformation Chamber has four main component's to helping transform lives and help heal people on many levels. The first being that while sitting inside The Transformation Chamber the persons body will be exposed to infrared and near infrared lighting, while also being guided by a guided meditation to help put the person into the parasympathetic nervous system. While sitting down and experiencing this infrared and near infrared lighting and meditating, their feet will be stimulated by vibration to help move their mitochondria around. Lastly, they will experience cryotherapy as the final part of the restorative/transformation part.

### BRIEF DESCRIPTION OF THE SEVERAL VIEWS OF THE DRAWING

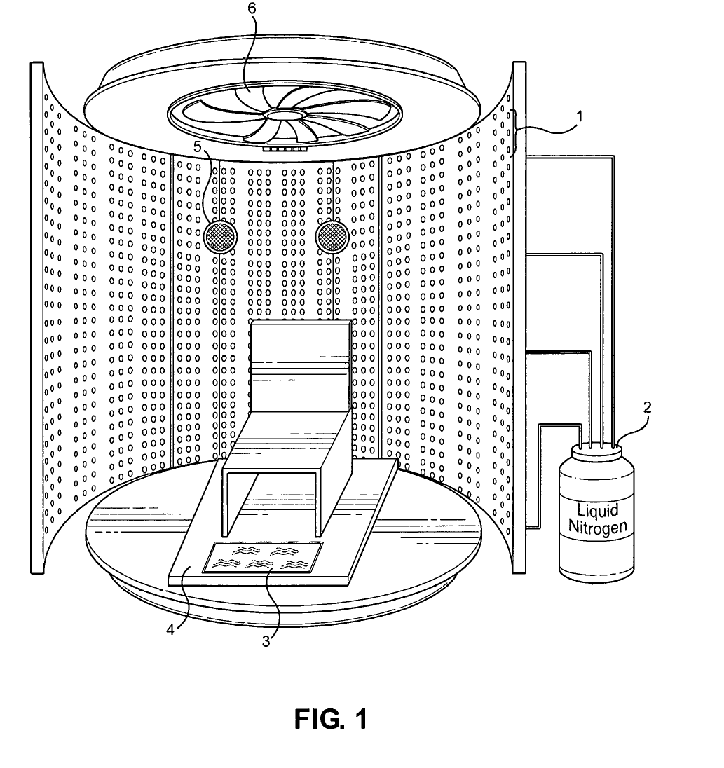

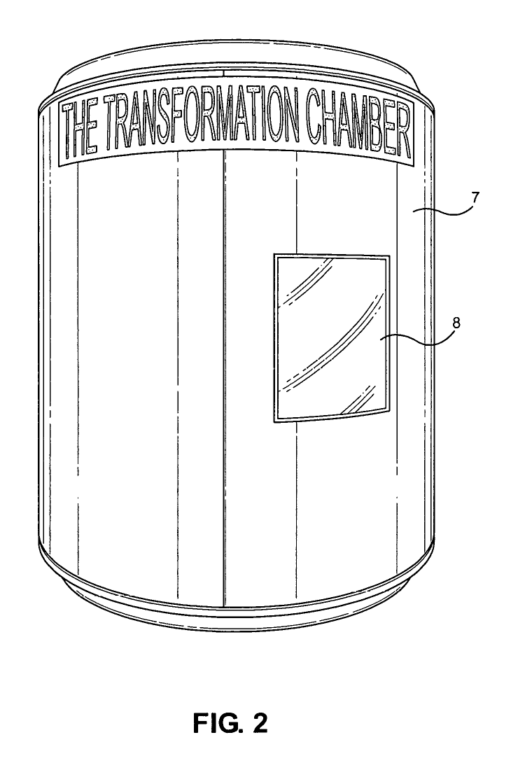

In FIG. 1 reference #1 displays infrared lighting, in FIG. 1 #2 displays liquid nitrogen for cryotherapy, in FIG. 1 reference #3 displays vibrating floor, in FIG. 1 reference #4 displays elevating floor, in FIG. 1 reference #5 displays speakers, in FIG. 1 reference #6 displays hinging fan, in FIG. reference #7 displays entrance/exit door, in FIG. 2 reference #8 displays tablet to start and stop Transformation Chamber

### DETAILED DESCRIPTION OF INVENTION

The Transformation Chamber stands upright at 100″×44″×44″ (8′ tall×3.5′ in each direction). The voltage of the chamber is 220-240VAC 11A max 50/60 Hz with a max consumption of 2400 watts. The Transformation Chamber is made of acrylics with red and near infrared l.e.d light system. The system will be switchable through a programmable tablet placed on outside of the chamber. Each panel of red and near infrared lights will consist of 4 row's of light's with 3 row's of light in each row. Number of Diodes Red: 1260 nm×2400 Red: 1320 nm×2400 NIR: 1700 nm×2400. Irradiance: 34 mW/cm2. There will be seat to sit on and a portion of the floor will vibrate with a range from 20-30 Hz. It will encompass a sound system with speakers for the guided meditation, and to be able to hear and speak to the client inside The Transformation Chamber. At the top of the chamber will be a 3000 CFM fan to help keep the bed and person cool. It will also involve a lift in order to lift the person's head out of the machine after red light therapy with meditation. The next phase will be the cryotherapy phase. The cryogenic gas will be liquid nitrogen and will be able to reach temperatures of −166F.

1. The Transformation Chamber can help rejuvenate and restore the body to a healthier state.
